(19) United States
(12) Patent Application Publication
(10) Pub. No.: US 2010/0311155 A1
(43) Pub. Date: Dec. 9, 2010

(54) METHOD FOR TREATMENT OF ORGANIC MATERIAL

(76) Inventors: Jorgen Ejlertsson, Rimforsa (SE); Jonas Högström, Storvreta (SE); Mark Easingwood, Uppsala (SE)

Correspondence Address: Capitol City TechLaw, PLLC, PO BOX 1210, VIENNA, VA, 22183, US

(21) Appl. No.: 12/746,172

(22) PCT Filed: Dec. 4, 2008
(86) PCT No.: PCT/EP2008/010274

§ 371 Date: Aug. 16, 2010

(30) Foreign Application Priority Data
Dec. 6, 2007 (SE) 0702721-2

(51) Int. Cl. C12M 1/33; B09B 3/00; G05D 7/00; B01J 19/10

(52) U.S. Cl. 435/290.4; 422/110; 422/184.1; 204/157.62

(57) ABSTRACT

The present invention relates to a disintegrating system for treatment of organic material comprising: multiple disintegrating units (31, 32; 41, 42; 51, 52, 53) each having an inlet (33, 34; 43, 44; 55, 56, 57) for receiving material A, B, C, and an outlet (37, 38; 47, 48; 61, 62, 63) for outputting treated material. A first inlet (33; 43; 55) of a first disintegrating unit (31; 41; 51) is configured to receive organic material A, and a first feedback pipe (35; 45; 58, 59) is connected between the outlet (37; 47; 61) of the first disintegrating unit and the inlet (34; 44; 56) of a second disintegrating unit. An outflow (39; 49; 64) of the disintegrating system (30; 40; 50) is connected to the outlet (37; 47; 61) of at least the first disintegrating unit (31; 41; 51), wherein the sum of the introduced material A, B, C is available at the outflow (39; 49; 64).

### TECHNICAL FIELD

The present invention relates to a method for treatment of organic material, especially when producing biogas using anaerobic digestion of the organic material, and to a system for treatment of organic material according to the method.

### BACKGROUND

It is well known in the prior art that ultrasonic treatment of organic material will decrease the viscosity with maintained dry solid contents, whereby a higher degree of dry solid contents may be obtained, e.g. in a feed line inputting organic material into a conventional biogas producing reactor tank, such as described the international publication WO 2004/016796 A1, assigned to Tekniska Verken i Linköping AB. Ultrasonic reactors are available from several manufacturer and distributors, such as Ultra\ddot{}Sonus, Sonica, Ultrawaves and Purac.

Ultrasonic treatment of organic material in a biogas producing system is presently used to increase the amount of biogas that may be produced. The organic material is mechanical broken before it is introduced into the reactor. The capacity of prior art ultrasonic equipment is rather limited, which makes it difficult to treat all the organic material before it is introduced into the reactor. FIG. 1 shows a prior art system 10 provided with two independent prior art systems for treating organic material when producing biogas in a tank reactor under anaerobic condition. In the first prior art system only x % (approx 30-40%) of an organic material introduced by a pipe 13 is subject to ultrasonic treatment in an ultrasonic reactor 11 before it is transported to a reactor tank 12. The dry solids contents is limited to 2-3% by weight TS, and the energy consumption needed to perform the ultrasonic treatment is approximately 4 W/h per litre. It is, however, possible to use several ultrasonic reactors in parallel but the amount of energy needed would result in a net loss of energy for the complete biogas producing process. Thus, the organic material is only partly treated by the ultrasonic reactor 11, and thereafter introduced through a feed pipe 15 into the reactor tank 12.

The limited capacity of the ultrasonic reactor makes it impossible to treat all the organic material before introducing it into the reactor which is a drawback. The second prior art system 16, e.g. disclosed in the international publication WO 2005/7016829, comprises a feedback loop including an ultrasonic reactor 17. Sludge from the reactor tank 12 is pumped and introduced into the ultrasonic reactor 17 for ultrasonic treatment, and is thereafter reintroduced into the reactor tank 12. Increased surface area of the organic material is achieved to increase the biogas production. Internal mixing is provided in the reactor tank 12, as exemplified by an agitator 14.

JP 2006-122875 discloses an equipment for sludge solubilisation treatment including a feedback loop from the outlet of an ultrasonic reactor to the inlet of the same ultrasonic reactor. At least a portion of the material available at the outlet is fed back to the inlet in order to decrease the viscosity of the material introduced. The capacity of the treatment equipment is increased compared to the prior art system described in WO 2005/7016829.

### SUMMARY OF THE INVENTION

An object with the present invention is to provide a system and a method for disintegrating at least one organic material to control the viscosity of the organic material while maintaining high dry solids contents of the organic material compared to the prior art.

The object is achieved by a system comprising at least two disintegrating units, such as ultrasonic reactors or mechanical dispersion devices, each provided with an inlet for receiving organic material, and an outlet for outputting disintegrated organic material. A first feedback pipe is connected between the outlet of one of the disintegrating units, which also is connected to an outflow of the system, and the inlet of another.

In a preferred embodiment, a second feedback pipe is provided to feedback the disintegrated organic material at the outlet of the another disintegrating unit to the inlet of the disintegrating unit being connected to the outflow.

An advantage with the present invention is that different organic material may be mixed together more easily for instance before it is introduced into a biogas producing system.

Another advantage of the present invention is that organic material with low dry solids contents may be subject to ultrasonic treatment compared to prior art systems.

Another advantage and aspect of the present invention is that an increased amount of biogas may be produced in an anaerobic digestion process for producing biogas compared to prior art systems.

Further objects and advantages will be apparent for a skilled person from the detailed description and the accompanying drawings.

### BRIEF DESCRIPTION OF THE DRAWINGS

FIG. 1 shows a system for producing biogas from organic material with ultrasonic treatment according to prior art.

FIG. 2a shows a variant of an ultrasonic system for treatment of organic material before being fed into a biogas producing system according to prior art.

FIG. 2b shows another variant of an ultrasonic system for treatment of organic material in a feedback loop arranged to a biogas producing system according to prior art.

FIG. 3 shows a first embodiment of a disintegrating system in which more than one type of organic material may be treated.

FIG. 4 shows a second embodiment of a disintegrating system in which more than one type of organic material may be treated.

FIG. 5 shows a third embodiment of a disintegrating system in which more than one type of organic material may be treated.

FIG. 6 shows a first embodiment of a biogas producing system including disintegrating systems according to the invention.

FIG. 7 shows a second embodiment of a biogas producing system including disintegrating systems according to the invention.

FIG. 8 shows an alternative embodiment of a biogas producing system in which material with different properties, such as different temperature or dry solids contents, are mixed before introduced into a disintegrating system.

### DETAILED DESCRIPTION OF PREFERRED EMBODIMENTS

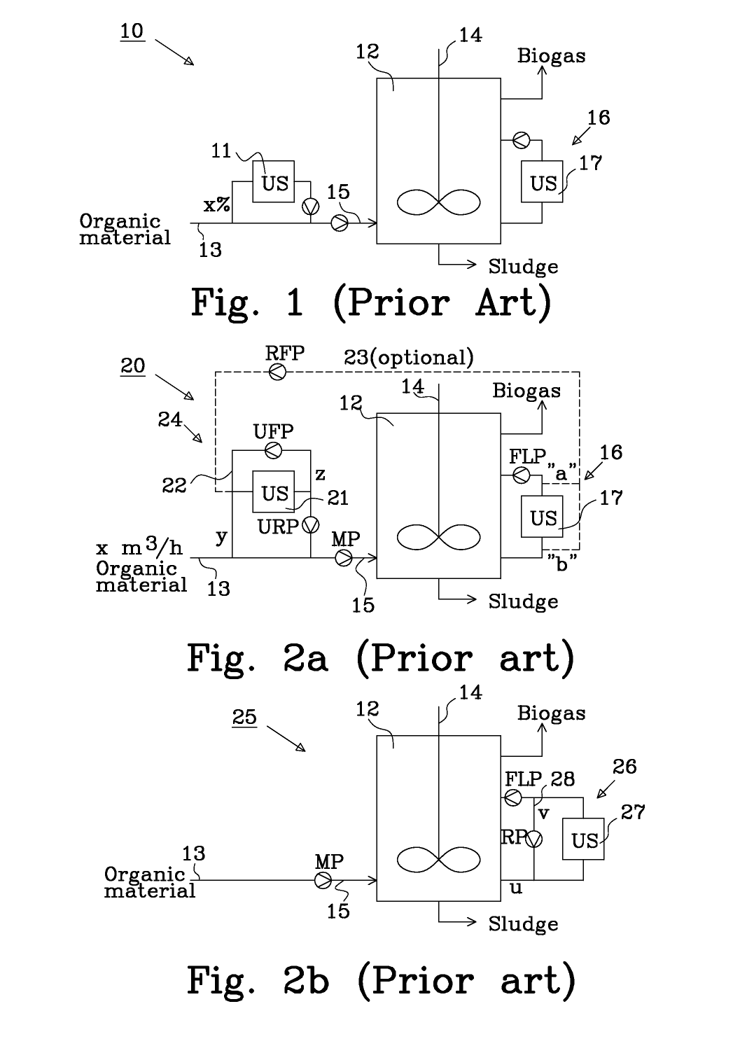

FIG. 1 discloses a biogas producing system 10 provided with two independent ultrasonic treatment system according to prior art, as described in more detail above.

FIG. 2a discloses a biogas producing system 20 provided with a variant of a prior art ultrasonic treatment system 24 for treatment of organic material. An amount (“x” m³/h) of organic material is fed into a pipe 13 and an ultrasonic reactor pump (URP) feeds a part “y” of the organic material fed into pipe 13 to an inlet of an ultrasonic reactor 21. The URP controls the amount, which preferably corresponds to 30-100% of “x”, and the remaining part of the organic material, preferably 0-70%, is directly introduced into the reactor tank 12 through the feed pipe 15. A feedback loop 22 is provided from an outlet of the ultrasonic reactor 21 to the inlet of the ultrasonic reactor 21. An amount “z” of the ultrasonic treated organic material present at the outlet is introduced into the feedback loop 22 using an ultrasonic feedback pump (UFP). The UFP controls the amount, which preferably corresponds to up to 1000% of “y”, more preferably 100-300% of “y”. The dry solid contents of the ultrasonic treated organic material is up to 6-8% TS. The outlet of the ultrasonic reactor is also connected to the inlet of the reactor tank 12 through the feed pipe 15. A main pump (MP) controls the amount of organic material “x” fed into the reactor tank 12, and thus the amount of organic material fed into the pipe 13.

The biogas producing system 20 is further provided with a prior art ultrasonic treatment system 16, as described in connection with FIG. 1, having a feedback loop including an ultrasonic reactor 17 and a feedback loop pump (FLP). Digested organic material, or sludge, from the reactor tank 12 is pumped, or let out using a valve (not shown), and introduced into the ultrasonic reactor 17 for ultrasonic treatment using the FLP, and is thereafter reintroduced into the reactor tank 12. An optional return feed pipe 23 (dashed line) may be provided, instead of the feedback loop 22, between the outlet of the ultrasonic reactor 17 (position “a”) and the inlet of the ultrasonic reactor 21. A return feed pump RFP may be provided. Ultrasonic treated sludge may then be returned and mixed with freshly introduced organic material provided through pipe 13. The feedback loop 22 may be provided together with the return feed pipe 23, whereby ultrasonic treated organic material provided through the feedback loop 22 is introduced into the ultrasonic reactor 21 together with organic material through pipe 13 and ultrasonic treated organic material from the ultrasonic reactor 17.

A constant flow over the ultrasonic reactor 21 is achieved, and the treatment effect is reduced, by circulating digested organic material, or sludge, from the reactor tank. The sludge may be ultrasonic treated, pumped from position “a”, or non-ultrasonic treated, pumped from position “b”.

FIG. 2b shows a second embodiment of an ultrasonic system 26 for treatment of organic material in a feedback loop provided with a ultrasonic reactor 27 arranged to a biogas producing system 25, similar to the prior art system 16 described in connection with FIG. 1. A feedback loop pump (FLP) is arranged in the feedback loop to pump, or to let out using a valve (not shown), a certain amount of digested organic material “u” from the reactor tank 12 and introduce the same amount of ultrasonic treated organic material into the reactor tank 12. The ultrasonic system 26 further comprises a return pipe 28 between the outlet and the inlet of the ultrasonic reactor 27. A return pump (RP) is provided in the return pipe and configured in such a way that a predetermined amount “v” of the ultrasonic treated organic material is fed back to the inlet of the ultrasonic reactor 27. The RP controls the amount, which preferably corresponds to up to 1000% of “u”, more preferably 100-300% of “u”.

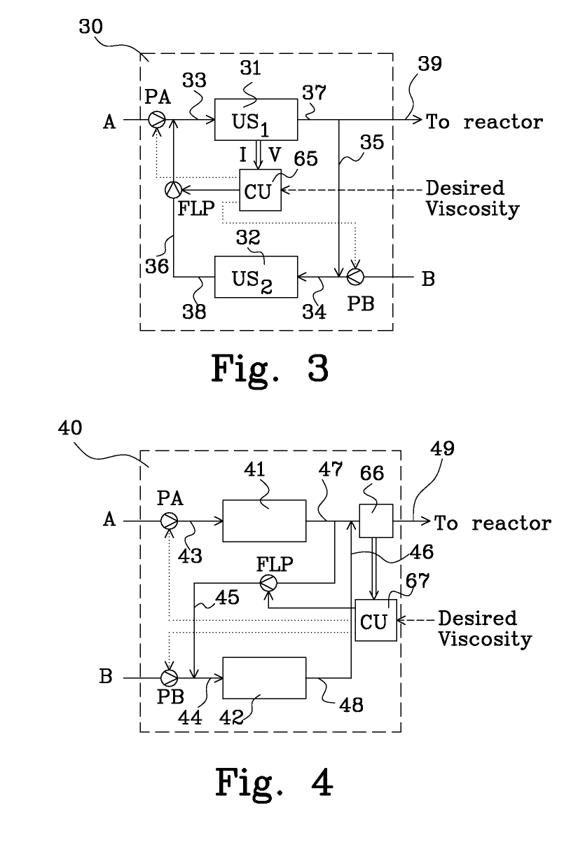

FIG. 3 shows a first embodiment of a disintegrating system according to the invention, which in this embodiment is an ultrasonic system 30 comprising two ultrasonic reactors 31 and 32. Organic material “A” is introduced into an inlet 33 of a first ultrasonic reactor 31 (denoted US₁ in FIG. 3) through a first feeding pipe, and organic material “B”, which may differ from organic material “A”, is introduced into an inlet 34 of a second ultrasonic reactor 32 (denoted US₂ in FIG. 3) through a second feeding pipe. Ultrasonic treated organic material is transferred from an outlet 37 of the first ultrasonic reactor US₁ to the inlet 34 of the second ultrasonic reactor US₂ through a first feedback pipe 35, and ultrasonic treated organic material is transferred from an outlet 38 of the second ultrasonic reactor US₂ to the inlet 33 of the first ultrasonic reactor US₁ through a second feedback pipe 36. Ultrasonic treated organic material is accessible for a biogas producing reactor tank at an outflow 39 connected to the outlet 37 of the first ultrasonic reactor US₁.

An advantage with this embodiment is that different organic material may be mixed together more easily for instance before it is introduced into a biogas producing system such as described in connection with FIG. 2a. This is achieved by controlling the flow through a number of pumps. A first feeding pump PA is provided to introduce organic material “A” into the first ultrasonic reactor 31, and a second feeding pump PB is provided to introduce organic material “B” into the second ultrasonic reactor 32. The capacity of each pump PA and PB is preferably 0-15 m³/h. A feedback loop pump FLP, which is arranged in the feedback pipe 36 has preferably a capacity of 15-30 m³/h.

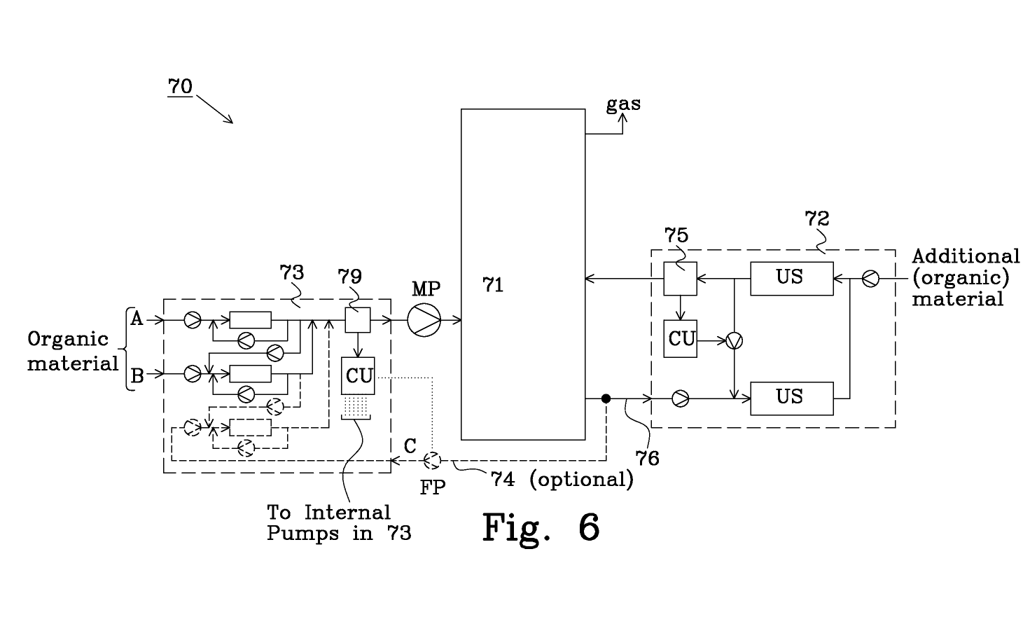

The amount of ultrasonic treated material that is transported to a biogas producing reactor through outflow 39 is controlled by a main pump (not shown) arranged at the inlet of the tank reactor as illustrated in connection with FIG. 6. If the main pump is set to transport Q m³/h into the biogas producing reactor, the sum of the organic materials A and B introduced through PA and PB should be equal to Q, i.e. A+B=Q. In order to maintain a loop flow, the flow through FLP needs to be higher than Q.

Viscosity is an interesting property of the ultrasonic treated material provided through the outflow 39 that might be desired to monitor. This may be achieved by a viscosity sensor arranged at the outflow 39 connected to a control unit, which in turn controls the flow through the FLP as illustrated in FIG. 4.

In FIG. 3, an alternative embodiment is shown wherein the viscosity sensor is omitted and the viscosity of the material transported through the first ultrasonic reactor 31 (and thus the material available at the outflow 39) may be determined by measuring the relationship between the voltage and current supplied to the ultrasonic reactor 31. The relationship between the voltage and current varies as a function of the viscosity of the treated organic material and is analysed in a control unit (CU) 65, which in turn controls the flow through the FLP. It is naturally possible to also control the flow of the first and second feeding pump PA and PB from the CU 65, as indicated by the dotted lines.

FIG. 4 shows a second embodiment of a disintegrating system according to the invention, which in this embodiment is a mechanical dispersion system 40 comprising two dispersion devices 41 and 42 arranged in a different way compared to FIG. 3. Organic material “A” is introduced into a first dispersion device 41 through a first feeding pipe to an inlet 43 of the first dispersion device 41, and organic material “B”, which may differ from organic material “A”, is introduced into a second dispersion device 42 through a second feeding pipe to an inlet 44 of the second dispersion device 42. Treated organic material is transferred from the first dispersion device 41 to the inlet 44 of the second dispersion device 42 through a feedback pipe 45, and treated organic material is transferred from an outlet 48 of the second dispersion device 42 to an outlet 47 of the first dispersion device 41 through a feed forward pipe 46. Treated organic material is accessible for a biogas producing reactor tank at an outflow 49 connected to the outlet 47 of the first dispersion device 41 and the outlet 48 of the second dispersion device 42.

Treated organic material is provided through the outflow 49 to be accessible to a biogas producing reactor by controlling the flow through a number of pumps. A first feeding pump PA is provided to introduce organic material “A” into the first dispersion device 41, and a second feeding pump PB is provided to introduce organic material “B” into the second dispersion device 42. The capacity of each pump PA and PB is preferably 0-15 m³/h. A feedback loop pump FLP, which is provided in the feedback pipe 45 has in this embodiment preferably a capacity of 0-15 m³/h.

The amount of ultrasonic treated material that is transported to a biogas producing reactor through the outflow 49 is controlled by a main pump (not shown) arranged at the inlet of the tank reactor as illustrated in connection with FIG. 6. If the main pump is set to transport Q m³/h into the biogas producing reactor, the sum of the organic materials A and B introduced through PA and PB should be equal to Q, i.e. A+B=Q. The flow through FLP will determine how much of the organic material “A” that will be subject to another treatment in the second dispersion device 42.

A viscosity sensor 66 is in this embodiment arranged at the outflow 49 and is connected to a control unit 67, which in turn controls the flow through the FLP. More viscosity sensors may naturally be provided in the dispersion system 40 in order to monitor the viscosity and ultimately to control the flow through each available pump as indicated by the dotted lines.

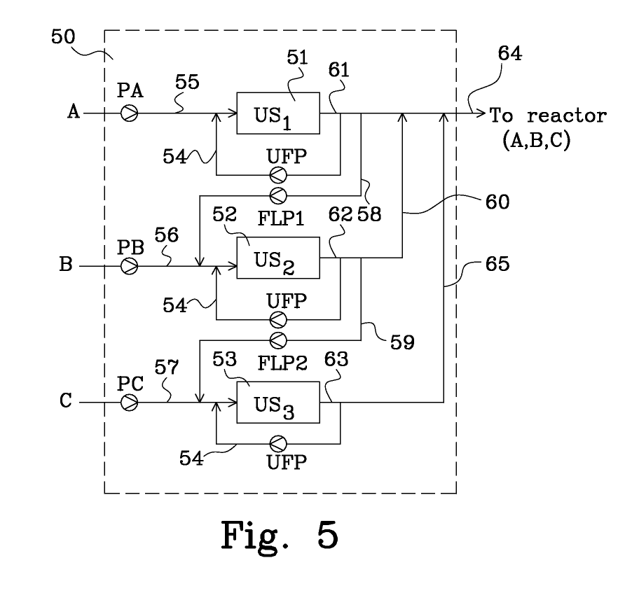

FIG. 5 shows a third embodiment of a disintegrating system according to the invention, which in this embodiment is an ultrasonic system 50 having a more complex structure. The ultrasonic system 50 comprises in this embodiment three ultrasonic reactors 51, 52 and 53 (denoted US₁, US₂ and US₃, respectively in FIG. 5). Each ultrasonic reactor is provided with a feedback loop 54 and an ultrasonic feedback pump UFP, as previously described in connection with FIG. 2a, and a feeding pipe to allow organic material “A”, “B” and “C”, respectively, to be introduced into an inlet 55, 56 and 57 of the respective ultrasonic reactor US₁, US₂ and US₃ using feeding pumps PA, PB and PC, respectively. Ultrasonic treated organic material from an outlet 61 of the first ultrasonic reactor US₁ is transferred to the inlet 56 of the second ultrasonic reactor US₂ through feedback pipe 58, and ultrasonic treated organic material from an outlet 62 the second ultrasonic reactor US₂ is transferred to the inlet 57 of the third ultrasonic reactor US₃ through feedback pipe 59. A first and a second feed forward pipe 60, 65 are provided from the outlet 62 of the second ultrasonic reactor and an outlet 63 of the third ultrasonic reactor, respectively, to the outlet 61 of the first ultrasonic reactor US₁ in order to transfer ultrasonic treated organic material to an outflow 64 connected to the outlet 61 of the first ultrasonic reactor US₁.

The capacity of the ultrasonic feedback pumps UFP is preferably 15-30 m³/h and the capacity of the feeding pumps PA, PB and PC is preferably 0-15 m³/h. Thus, the flow through the feedback loop 54 is at least equal to the flow through each feeding pump, and more preferably twice as high or more. Feedback loop pumps FLP are provided in each feedback pipe 58 and 59, and the capacity of the FLP is preferably 0-15 m³/h.

Organic material having higher dry solid contents may be fed into the ultrasonic reactors compared to the embodiment described in connection with FIG. 4 due to the presence of the ultrasonic feedback loop.

FIG. 6 shows a first embodiment of a biogas producing system 70 including a reactor tank 71 and disintegrating systems 72 and 73, such as described in connection with FIGS. 3-5, in a feedback loop and pretreatment for treatment of organic material in connection with producing biogas. It should be noted that the biogas producing system may be implemented with only a feedback loop or a pre-treatment system, as well as a combination of both.

The feedback loop comprises a first disintegrating system 72, similar to the system described in connection with FIG. 3 with the addition of an optional feedback pipe 74 from the outlet 76 of the reactor tank 71 to the pretreatment of the organic material. A viscosity sensor 75 is also used to control the pumps within the disintegrating system 72. Additional organic material from other processes may be introduced as indicated in the drawing. If the feedback loop is implemented without pre-treatment of the organic material, the feedback pipe 74 is not needed.

The pre-treatment system comprises a second disintegrating system 73, similar to the system described in connection with FIG. 4 or 5 (if the optional feedback pipe 74 is present). Organic material A and B are introduced and treated (using mechanical dispersion or ultrasound as described above) within the disintegrating system 73, and material C from the outlet 76 is introduced to the disintegrating system. A main pump MP feeds an appropriate amount of treated material into the reactor tank 71. A viscosity sensor 79 and control unit CU within the disintegrating system controls the pump activity within the disintegrating system 73, and optionally also the FP pump controlling the amount of material in the feedback pipe 74, in order to output organic material with a desired viscosity to the tank reactor 71.

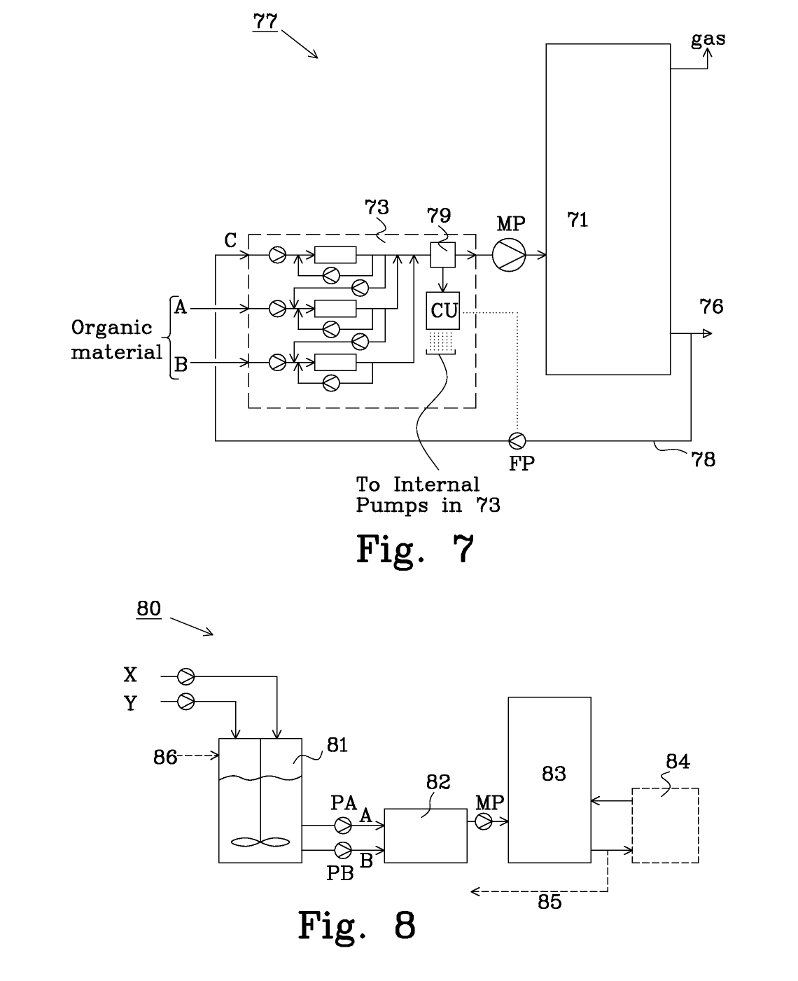

FIG. 7 shows a second embodiment of a biogas producing system 77, including a reactor tank 71 and a disintegrating system 73, similar to the system disclosed in connection with FIG. 5.

The system comprises a feedback loop 78 from the outflow 76 of the tank reactor 71. The flow is controlled by a feedback pump FP and introduced into the first disintegrating unit in the disintegrating system 73. The purpose for feeding sludge from the tank reactor 71 to the first disintegrating unit is to provide organic material with a suitable viscosity in the system if other organic material A and B, which are introduced to the second and third disintegrating unit, have a viscosity that make them difficult to move through the respective disintegrating unit by themselves.

The disintegrating system preferably comprises a viscosity sensor 79 and a control unit configured to control the internal pumps in the disintegrating unit as well as the feedback pump FP.

FIG. 8 shows an alternative embodiment of a biogas producing system 80 in which material “X” and “Y” with different properties, such as different temperature or dry solids contents, are mixed in a premixing vessel 81 before introduced into a disintegrating system 82. In this example two feed pipes with pumps PA and PB are used to introduce organic material “A” and “B” into the disintegrating system 82.

The organic material “A” and “B” are in this case the same and consists of a mixture of “X” and “Y”. Optionally, nutriments may also be introduced into the premixing vessel 81 as indicated by the dashed arrow 86. A main pump MP will feed treated material from the disintegrating system 82 into a tank reactor 83.

It is also possible to add a feedback loop 84, as described in connection with FIG. 6, to the system in FIG. 7. A feedback pipe 85 may be implemented to feed material from an outlet of the tank reactor 83 either to the disintegrating system (as described in connection with FIG. 6) or as one of the materials “Y” introduced into the premixing vessel 81. “X” in this case would be non-treated organic material may have a different temperature, or dry solids contents, compared to the material obtained from the outlet of the tank reactor 83. The premixing vessel 81 will equalize any difference and might increase the efficiency of the disintegrating system.

In an alternative embodiment, the disintegrating system 82 may include a prior art disintegrating system, as disclosed in JP 2006-122875 and described in connection with FIGS. 2a and 2b, with the addition of a viscosity detector, similar to that described in connection with FIG. 3 or 4, arranged at the outflow of the disintegrating system 82. A control unit attached to the viscosity detector will control the amount of organic material fed back to the inlet of the disintegrating unit (e.g. ultrasonic reactor) and thereby also the viscosity of the organic material available to be introduced into the reactor tank 83. In this case, only one inlet of organic material, e.g. “A”, from the pre-mixing tank 81 is necessary.

Although the introduced material has been exemplified in connection with FIGS. 3-7 as organic material “A”, “B” and “C”, which is generally preferred, the scope of the invention should not be limited to this since it might be desirable to introduce non-organic material into the system. However, in order to produce disintegrated organic material, at least one of the materials introduced has to be organic, and nutriments that could be used in a digestion process, such as Iron, Cobalt, Nickel, Selenium, Tungsten, Boron, Molybdenum, and Vanadium, may be introduced to be mixed with the organic material in the system, or the nutriments may be pre-mixed with organic material before it is introduced.

The pumps used in all the described embodiments of the invention are preferably eccentric screw pumps or chopper pumps, obtainable from e.g. ITT Flygt, KSB or Scanpump.

The terminology “disintegrating unit” is used in the claims as a generic term for ultrasonic reactor, mechanical dispersion device, as well as any other type of device that will disintegrate a material.

Furthermore, the disintegrating units of the described disintegrating units in connection with FIGS. 3-8 are of the same type, i.e. either ultrasound reactors or mechanical dispersion devices, but the invention should not be limited to this. It is possible to construct a disintegrating system having a mechanical dispersion device as a first integrating unit and an ultrasound reactor as a second disintegrating system.

The capacity and operating characteristics of each pump is selected to transport the desired amount of organic material through each ultrasonic reactor, and to create a non-laminar flow through each ultrasonic reactor. It is preferred that the flow through each ultrasonic reactor is above 0.5 m/s in order to avoid an undesired laminar flow.

It should be noted that although the described embodiment above illustrates a biogas producing system having a feedback loop of treated digested sludge being fed back to the same tank reactor, as described in connection with FIG. 6, other configurations of tank reactors are possible. Tank reactors may be arranged in parallel and/or in series with each other, whereby digested sludge from a first tank reactor may be pumped into a disintegrating system and the treated digested sludge is thereafter delivered to a second tank reactor and/or delivered back to the first tank reactor.

1. A disintegrating system for treatment of organic material comprising:
multiple disintegrating units, each of the disintegrating units being an ultrasonic reactor or a mechanical dispersion device, and each of the disintegrating units having an inlet for receiving material, and an outlet for outputting treated material, wherein the inlet of a first disintegrating unit of the multiple disintegrating units is configured to receive organic material,
a first feedback pipe connected between the outlet of the first disintegrating unit and the inlet of a second disintegrating unit of the multiple disintegrating units, and
an outflow connected to the outlet of at least the first disintegrating unit, wherein the sum of the introduced material is available at the outflow.

2. The disintegrating system according to claim 1, further comprising a feedback loop arranged to feed back at least a part of the treated material at the outlet of each disintegrating unit to the inlet of the same disintegrating unit.

3. The disintegrating system according to claim 1, wherein a second feedback pipe is connected between the outlet of the second disintegrating unit and the inlet of the first disintegrating unit.

4. The disintegrating system according to claim 3, wherein
a first feeding pump is provided to feed organic material to the inlet of the first disintegrating unit,
a second feeding pump is provided to feed material to the inlet of the second disintegrating unit, and
a feedback loop pump is provided in the second feedback pipe, the flow capacity of the feedback loop pump is at least equal to the flow capacity of each of the first and the second feeding pumps.

5. The disintegrating system according to claim 1, wherein the outflow is connected to the outlet of only the first disintegrating unit.

6. The disintegrating system according to claim 1, wherein a feed forward pipe is connected between the outlet of the second disintegrating unit and the outlet of the first disintegrating unit.

7. The disintegrating system according to claim 6, wherein
a second feedback pipe is connected between the outlet of the second disintegrating unit and the inlet of a third disintegrating unit, and
a second feed forward pipe is connected between the outlet of the third disintegrating unit and the outlet of the first disintegrating unit.

8. The disintegrating system according to claim 6, wherein the outflow is connected to the outlet of multiple disintegrating units.

9. The disintegrating system according to claim 1, wherein
a viscosity detector is provided to determine the viscosity of the treated material at the outlet of the first disintegrating unit, and
a control unit is configured to receive measured parameters from the viscosity detector to control the flow through the first feedback pipe.

10. The disintegrating system according to claim 9, wherein the viscosity detector is one of:
a viscosity sensor placed at the outflow; and
an analyzer provided with voltage and current supplied to the first disintegrating unit.

11. A biogas producing system using anaerobic digestion of organic matter, the system comprising:
a tank reactor provided with an inlet for receiving organic material suitable for biogas production, the tank reactor containing biogas-producing bacteria for digestion under anaerobic conditions,
feeding means to feed the organic material into the tank reactor to obtain digestion while producing biogas and forming digested sludge,
at least one disintegrating system configured to feed treated organic material to the inlet of the tank reactor, the disintegrating system including
multiple disintegrating units, each of the disintegrating units being an ultrasonic reactor or a mechanical dispersion device, and each of the disintegrating units having an inlet for receiving material, and an outlet for outputting treated material, wherein the inlet of a first disintegrating unit of the multiple disintegrating units is configured to receive organic material,
a first feedback pipe connected between the outlet of the first disintegrating unit and the inlet of a second disintegrating unit of the multiple disintegrating units, and
an outflow connected to the outlet of at least the first disintegrating unit, wherein the sum of the introduced material is available at the outflow.

12. The biogas producing system according to claim 11, further comprising a premixing vessel configured to receive at least two materials with different properties, and configured to output premixed materials to one of the at least one disintegrating systems.

13. A method for treatment of organic material in a disintegrating system with multiple disintegrating units, each of the disintegrating units having an inlet for receiving material, and an outlet for outputting treated material, the method comprising:
providing each of the disintegrating units as an ultrasonic reactor or a mechanical dispersion device,
introducing organic material at the inlet of a first disintegrating unit of the multiple disintegrating units,
providing treated organic material from the outlet of at least the first disintegrating unit to an outflow of the disintegrating system,
connecting a first feedback pipe between the outlet of the first disintegrating unit and the inlet of a second disintegrating unit of the multiple disintegrating units, and
providing the sum of the introduced material at the outflow.

14. The method according to claim 13, further comprising arranging a feedback loop to feed back at least a part of the treated material at the outlet of each disintegrating unit to the inlet of the same disintegrating unit.

15. The method according to claim 13, further comprising connecting a second feedback pipe between the outlet of the second disintegrating unit and the inlet of the first disintegrating unit.

16. The method according to claim 15, further comprising:
providing a first feeding pump to feed organic material to the inlet of the first disintegrating unit,
providing a second feeding pump to feed material to the inlet of the second disintegrating unit, and
providing a feedback loop pump in the second feedback pipe, and selecting the flow capacity of the feedback loop pump to be at least equal to the flow capacity of each of the first and the second feeding pumps.

17. The method according to claim 13, further comprising connecting the outflow to the outlet of only the first disintegrating unit.

18. The method according to claim 13, further comprising connecting a feed forward pipe between the outlet of the second disintegrating unit and the outlet of the first disintegrating unit.

19. The method according to claim 18, further comprising:
connecting a second feedback pipe between the outlet of the second disintegrating unit and the inlet of a third disintegrating unit, and
connecting a second feed forward pipe between the outlet of the third disintegrating unit and the outlet of the first disintegrating unit.

20. The method according to claim 18, further comprising connecting said outflow to the outlet of multiple disintegrating units.

21. The method according to claim 13, further comprising:
providing a viscosity detector to determine the viscosity of the treated material at the outlet of the first disintegrating unit, and
configuring a control unit to receive measured parameters from the viscosity detector to control the flow through the first feedback pipe.

22. The method according to claim 21, further comprising selecting the viscosity detector from one of:
a viscosity sensor placed at the outflow; and
an analyzer provided with voltage and current supplied to the first disintegrating unit.
